(19) United States
(12) Patent Application Publication
(10) Pub. No.: US 2013/0237688 A1
(43) Pub. Date: Sep. 12, 2013

(54) MULTIMODALITY AGENTS FOR TUMOR IMAGING AND THERAPY

(71) Applicants: HEALTH RESEARCH, INC., Buffalo, NY (US); THE RESEARCH FOUNDATION OF STATE UNIVERSITY OF NEW YORK, Amherst, NY (US)

(72) Inventors: Ravindra K. Pandey, East Amherst, NY (US); Suresh Pandey, Buffalo, NY (US); Lalit Goswami, Columbia, MO (US); Allan Oseroff, Buffalo, NY (US); Shipra Dubey, Williamsville, NY (US); Munawwar Sajjad, Clarence Center, NY (US)

(73) Assignees: The Research Foundation of State University of New York, Amherst, NY (US); Health Research, Inc., Buffalo, NY (US)

(21) Appl. No.: 13/863,145

(22) Filed: Apr. 15, 2013

Related U.S. Application Data
(62) Division of application No. 12/677,381, filed on Nov. 23, 2010, now abandoned.
(60) Provisional application No. 60/993,910, filed on Sep. 14, 2007.

(51) Int. Cl. C07K 7/06

(52) U.S. Cl. CPC C07K 7/06
USPC 530/330

(57) ABSTRACT

A compound that is a conjugate of an antagonist to an integrin expressed by a tumor cell and at least one of a tumor avid tetrapyrollic photosensitizer, a fluorescent dye, and a radioisotope labeled moiety wherein the radioisotope is ¹¹C, ¹⁸F, ⁶⁴Cu, ¹²⁴I, ⁹⁹Tc, ¹¹¹In or GdIII and its method of use for diagnosing, imaging and/or treating hyperproliferative tissue such as tumors. Preferably the photosensitizer is a tumor avid tetrapyrollic photosensitizer, e.g. a porphyrin, chlorin or bacteriochlorin, e.g. pheophorbides and pyropheophorbides. Such conjugates have extreme tumor avidity and can be used to inhibit or completely destroy the tumor by light absorption. The integrin is usually αvβ3, α5β1, αvβ5, α4β1, or α2β1. Preferably, the antagonist is an RGD peptide or another antagonist that may be synthetic such as a 4-{2-(3,4,5,6-tetra-hydropyrimidin-2-ylamino)ethyloxy}-benzoyl]amino-2-(S)-amino-ethyl-sulfonylamino group. Such compounds provide tumor avidity and imaging ability thus permitting selective and clear tumor imaging.

This application is a Divisional of U.S. application Ser. No. 12/677,381, filed Nov. 23, 2010, which is the National Stage of International Application No. PCT/US2008/010609, filed Sep. 11, 2008, which was published in English; said international Application claims priority from Application No. 60/993,910, filed Sep. 14, 2007.

### BACKGROUND OF THE INVENTION

Photodynamic therapy (PDT) is an effective local therapy based on a tumor localizing photosensitizer (PS) activated by long wavelength light directed at the treatment site. Current photosensitizers have high tumor selectivity, and light can be delivered almost anywhere in the body by thin, flexible optical fibers.

Tetrapyrollic photosensitizers, e.g. porphyrins including chlorins, bacteriochlorins and other porphyrin based derivatives, including their analogs and derivatives, have recently found superior utility as photodynamic compounds for use in diagnosis and treatment of disease, especially certain cancers and other hyperproliferative diseases such as macular degeneration. These compounds have also found utility in treatment of psoriasis and papillomatosis.

Such derivatives include dimers and trimers of these compounds. Permissible derivatives also include ring variations of these compounds; provided that, the central sixteen sided four nitrogen heterocycle of these compounds remains intact. Chlorophyllins, purpurins, pheophorbides, and their derivatives are, therefore, included within “porphyrins, chlorins, and bacteriochlorins and their derivatives and analogs”. Such derivatives include modifications of substituents upon these ring structures, e.g. pyropheophorbides.

Numerous articles have been written on this subject, e.g. “Use of the Chlorophyll Derivative Purpurin-18, for Synthesis of Sensitizers for Use in Photodynamic Therapy”, Lee et al., J. Chem. Soc., 1993, (19) 2369-77; “Synthesis of New Bacteriochlorins And Their Antitumor Activity”, Pandey et al., Biology and Med. Chem. Letters, 1992; “Photosensitizing Properties of Bacteriochlorophyllin a and Bacteriochlorin a, Two Derivatives of Bacteriochlorophyll a”, Beems et al., Photochemistry and Photobiology, 1987, v. 46, 639-643; “Photoradiation Therapy. II. Cure of Animal Tumors With Hematoporphyrin and Light”, Dougherty et al., Journal of the National Cancer Institute, July 1975, v. 55, 115-119; “Photodynamic therapy of C3H mouse mammary carcinoma with hematoporphyrin di-esters as sensitizers”, Evensen et al., Br. J. Cancer, 1987, 55, 483-486; “Substituent Effects in Tetrapyrrole Subunit Reactivity and Pinacol-Pinacolone Rearrangements: VIC-Dihydroxychlorins and VIC-Dihydroxybacteriochlorins” Pandey et al., Tetrahedron Letters, 1992, v. 33, 7815-7818; “Photodynamic Sensitizers from Chlorophyll: Purpurin-18 and Chlorin p₆ ”, Hoober et al., 1988, v.48, 579-582; “Structure/Activity Relationships Among Photosensitizers Related to Pheophorbides and Bacteriopheophorbides”, Pandey et al., Bioorganic and Medicinal Chemistry Letters, 1992, v 2, 491-496; “Photodynamic Therapy Mechanisms”, Pandey et al., Proceedings Society of Photo-Optical Instrumentation Engineers (SPIE), 1989, v 1065, 164-174; and “Fast Atom Bombardment Mass Spectral Analyses of Photofrin II@ and its Synthetic Analogs”, Pandey et al., Biomedical and Environmental Mass Spectrometry, 1990, v. 19, 405-414. These articles are incorporated by reference herein as background art.

Numerous patents in this area have been applied for and granted world wide on these photodynamic compounds. Reference may be had, for example to the following U.S. Patents which are incorporated herein by reference: U.S. Pat. Nos. 4,649,151; 4,866,168; 4,889,129; 4,932,934; 4,968,715; 5,002,962; 5,015,463; 5,028,621; 5,145,863; 5,198,460; 5,225,433; 5,314,905; 5,459,159; 5,498,710; and 5,591,847.

One of these compounds “Photofrin®” has received approval for use in the United States, Canada and Japan. Others of these compounds have also received at least restricted approval, e.g. BPD for treatment of macular degeneration and others are in clinical trials or are being considered for such trials.

The term “porphyrins, chlorins and bacteriochlorins” as used herein is intended to include their derivatives and analogs, as described above, and as described and illustrated by the foregoing articles and patents incorporated herein by reference as background art.

Such compounds have been found to have the remarkable characteristic of preferentially accumulating in tumors rather than most normal cells and organs, excepting the liver and spleen. Furthermore, many such tumors can be killed because the compounds may be activated by light to become tumor toxic.

Such compounds are preferentially absorbed into cancer cells, and destroy cancer cells upon being exposed to light at their preferential wavelength absorbance near infrared (NIR) absorption. Further such compounds emit radiation at longer wavelengths than the preferential absorption wavelength, such that light penetrates several centimeters of tissue. It is thus possible to sense and quantitate photosensitizer concentration in subsurface tissues from measurements of diffuse light propagation.

However, for small, bulky, or buried lesions, it may be difficult to detect the malignancies and/or to properly place the optical fibers to illuminate the full extent of the tumor. Therefore the approach of guided therapy utilizing highly selective optical and radionuclide tumor imaging, allowing tumor visualization, image-guided placement of the optical fibers, and subsequent photodynamic destruction of the lesions would be extremely useful in cancer diagnosis and therapy.

Optical imaging is a rapidly evolving field. Optical contrast agents can provide planar and tomographic images with high sensitivity. For small animals, planar images are adequate, but optical tomographic reconstruction of fluorescence images is becoming feasible.

Most of the porphyrin-based photosensitizers (PS) fluoresce, and the fluorescence properties of these porphyrins in vivo has been exploited by several investigators for detection of early-stage cancers in the lung, bladder and various other sites, and to guide the activating light for treatment. However, PS are not optimal fluorophores for tumor detection or treatment guidance: (1) They have weak fluorescence compared to cyanine dyes. They have small Stokes shifts, making it difficult to separate the fluorescence from excitation light.

Fluorescent cyanine dyes with NIR excitation and emission wavelengths can have high quantum yields and excitation coefficients, and appropriate Stokes shifts. They have high extinction coefficients and appropriate Stokes shifts. We have determined that such compounds coupled with photosensitizers can be used as “Bifunctional Agents” (i. e. tumor imaging and phototherapy). See e.g. copending PCT Patent Application PCT/US05/24782.

Positron emission tomography (PET) predominately has been used to image and assay biochemical processes and circular function. However, there has been growing use of radiolabeled peptide ligands to target malignancies. Available isotope labels include ¹¹C (t_{1/2}=20.4 min) ¹⁸F (t_{1/2}=110 min), ⁶⁴Cu (t_{1/2}=12.8 h and ¹²⁴I (t_{1/2}=4.2 days). For targeting photosensitizers, a long circulation time may be desired, as it can increase delivery of the agent into tumors. We have shown that 1-124 labeled photosensitizers can be used for PET imaging and PDT. See e.g. copending U.S. patent application Ser. No. 11/353,626 filed Feb. 14, 2006.

Integrins are heterodimeric transmembrane adhesion receptors that play an important role in cell-surface mediated signaling. There are at least 24 distinct integrin receptors identified, which are assembled from 18α and 8β subunits. αvβ3, α5β1, αvβ5, α4β1, α2β1 are known integrins expressed by tumor cells. As an example in accordance with the invention, integrin αvβ3 is used to illustrate the invention with binding to an RGD peptide, a small peptide containing an RGD sequence [arginine(Arg)-glycine(Gly)-aspartic acid(Asp) triamino acid sequence] It is understood that longer sequences, e.g. up to ten or more amino acids, may be used containing the RGD sequence and all such peptides are referred to herein as RGD peptides. As an example of non-peptide antagonists or ligands compounds containing a 4-{2-(3,4,5,6-tetrahydropyrimidin-2-ylamino)ethyloxy}-benzoyl]amino-2-(S)-aminoethylsulfonylamino (THPAB) group are used. We are initially focusing on the specific receptor, Integrin αvβ3, as an example of such Integrins expressed by tumor cells. Integrin αvβ3 is known for its high expression in tumor cells (3) and its binding with RGD peptides.

Sequence analysis of integrin αv subunit from various organisms (human, mouse, bull, chicken, frog, zebrafish) using both T-Coffee and ClustalW multiple sequence alignment programs shows high degree of their conservations, especially among the mammals. Similar results are also observed from the sequence analysis of the integrin β3.subunit from various organisms (human, mouse, rat, chicken, frog, zebrafish). Strict conservation of the implicated ligand binding residues is clearly observed.

As for 3D structures of integrins, several crystal structures are available at PDB. For Integrin β3 subunit, there are crystal structures of Integrin β3—Talin chimera complex (1MK7,1MK9), NMR structure of the Integrin β3 cytoplasmic domain (1S4X), as well as the Integrin αIIbβ3 receptor crystal (1TXV, 1TY3, 1TY5, 1TY6, 1TY7, 1TYE) and NMR (1M8O) structures. For the Integrin αvβ3 system, the structures of the extracellular domain of Integrin αvβ3 (1JV2) as well as its complex with Mn2+ (1M1X) and with the RGD ligand (1L5G) are available. In addition, recently the N-terminal PSI (plexin-semaphorin-integrin) domain of the β subunit structure has been reported in the context of the αvβ3 receptor (1U8C). We performed a pair-wise comparison of overall structure of integrin αvβ3 and αIIbβ3. It clearly shows the conservation of ion binding residues.

Crystal structure of integrin αvβ3 RGD peptide complex was carefully examined. The RGD peptide binds at the interface of αv and β3 subunits where an intricate network of interactions involving 3 Mn cations plays an important role in recognition of RGD Asp residue (See FIGS. 1 and 2).

Integrins are a major group of cell membrane receptors with both adhesive and signaling functions. They influence behavior of neoplastic cells by their interaction with the surrounding extracellular matrix, participating in tumor development. An increase in its expression is correlated with increased malignancy. Significant over expression of αvβ3 is reported in colon, lung, pancreas and breast carcinomas, and the expression of integrin was significantly higher in tumors of patients with metastases than in those without metastases.

The following references are incorporated herein as background art.
1. Yihui Chen, Amy Gryshuk, Samuel Achilefu, Tymish Ohulchansky, William Potter, Tuoxiu Zhong, Janet Morgan, Britton Chance, Paras N. Prasad, Barbara W. Henderson, Allan Oseroff and Ravindra K. Pandey, A Novel Approach to a Bifunctional Photosentizer for Tumor Imaging and Phototherapy. Bioconjugate Chemistry, 2005, 16, 1264-1274.
2. Suresh K. Pandey, Amy L. Gryshuk, Munawwar Sajjad, Xiang Zheng, Yihui Chen, Mohei M. Abouzeid, Janet Morgan, Ivan Charamisinau, Hani A. Nabi, Allan Oseroff and Ravindra K. Pandey, Multiomodality Agents for Tumor Imaging (PET, Fluorescence) and Photodynamic Therapy: A Possible See and Treat Approach. J. Med. Chem. 2005, 48, 6286-6295.
3. Xiaoyuan C. et al. Integrin avb3-Targeted Imaging of Lung Cancer. Neoplasia, 2005, 7, 271-279. Yihui Chen, Amy Gryshuk, Samuel Achilefu, Tymish Ohulchansky, William Potter, Tuoxiu Zhong, Janet Morgan, Britton Chance, Paras N. Prasad, Barbara W. Henderson, Allan Oseroff and Ravindra K. Pandey, A Novel Approach to a Bifunctional Photosentizer for Tumor Imaging and Phototherapy. Bioconjugate Chemistry, 2005, 16, 1264-1274.
4. Suresh K. Pandey, Amy L. Gryshuk, Munawwar Sajjad, Xiang Zheng, Yihui Chen, Mohei M. Abouzeid, Janet Morgan, Ivan Charamisinau, Hani A. Nabi, Allan Oseroff and Ravindra K. Pandey, Multiomodality Agents for Tumor Imaging (PET, Fluorescence) and Photodynamic Therapy: A Possible See and Treat Approach. J. Med. Chem. 2005, 48, 6286-6295.
5. Xiaoyuan C. et al. Integrin avb3-Targeted Imaging of Lung Cancer. Neoplasia, 2005, 7, 271-279.

### BRIEF DESCRIPTION OF THE DRAWINGS

FIG. 1 shows a crystal structure of integrin RGD peptide complex. A flat arrow indicates for β strand and a cylinder for a helix. White color is used for αv subunit and a porphyrin, chlorin or bacteriochlorin, e.g. pheophorbides and pyropheophorbides gray color for β3 subunit. Integrin RGD peptide, Arg-Gly-Asp-D-Phe-N-methyl Val is located between αv and β3 subunits shown in ball and stick figure. The Mn ions located near the RGD peptide are shown as spheres.

FIG. 2 shows how Asp interacts with residues from β3 subunit and Mn ions embedded in β3 subunit. Especially, the middle Mn ion is directly coordinated with Asp side chain (COO—) group. In turn, this Mn ion is coordinated by Ser 121, Ser 123, and Glu 220. These residues in turn are coordinated to two other Mn ions, which form additional coordination with other residues from β3 subunit. Asp side chain of RGD peptide also make a direct interaction with Asn 215. This network of interaction involving 3 Mn ions seems to be a very important stabilizing factor.

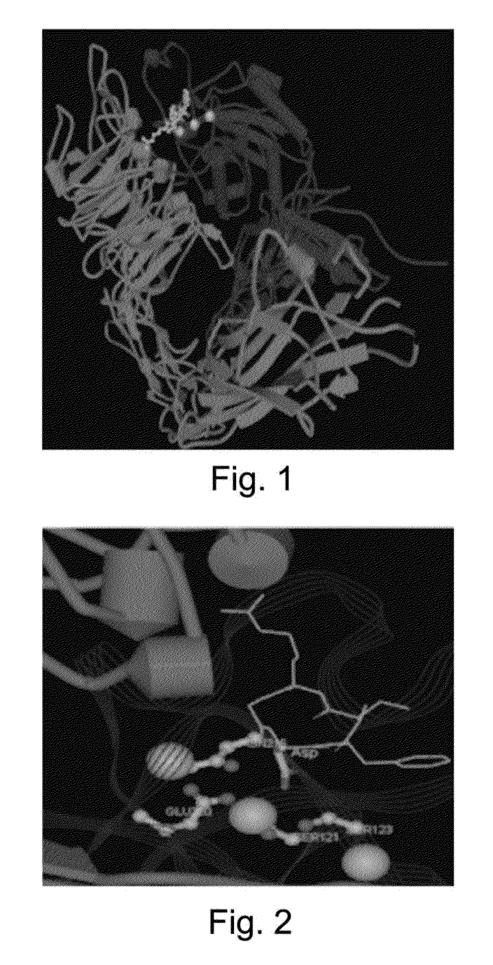

### BRIEF DESCRIPTION OF THE INVENTION

The invention is a compound that is a conjugate of an antagonist to an integrin expressed by a tumor cell and at least one of a fluorescent dye, or a tumor avid tetrapyrollic photosensitizer, that may be complexed with an element X where X is a metal selected from the group consisting of Zn, In, Ga, Al, or Cu or a radioisotope labeled moiety wherein the radioisotope is selected from the group consisting of ¹¹C, ¹⁸F, ₆₄Cu, ¹²⁴I, ⁹⁹Tc, ¹¹¹In and GdIII and its method of use for diagnosing, imaging and/or treating hyperproliferative tissue such as tumors and other uncontrolled growth tissues such as found in macular degeneration.

In a preferred embodiment, the compound is a tumor avid tetrapyrollic photosensitizer compound conjugated with an antagonist for an integrin expressed by a tumor cell. Such compounds have extreme tumor avidity and can be used to inhibit or completely destroy the tumor by light absorption. The tetrapyrollic photosensitizer is usually a porphyrin, chlorin or bacteriochlorin including pheophorbides and pyropheophorbides and the integrin is usually an αvβ3, α5β1, αvβ5, α4β1, or α2β1 integrin.

In a preferred embodiment, the antagonist is an RGD peptide or another antagonist that may be synthetic such as a 4-{2-(3,4,5,6-tetra-hydropyrimidin-2-ylamino)ethyloxy}-benzoyl]amino-2-(S)-aminoethyl-sulfonylamino group. The integrin is most commonly αvβ3.

The antagonist may be combined with an imaging compound such as a fluorescent dye or a structure including an element X where X is a metal selected from the group consisting of Zn, In, Ga, Al, or Cu or a radioisotope labeled moiety wherein the radioisotope is selected from the group consisting of ¹¹C, ¹⁸F, ⁶⁴Cu, ¹²⁴I, ⁹⁹Tc, ¹¹¹In. Such compounds provide tumor avidity and imaging ability thus permitting selective and clear tumor imaging.

Objects of this invention include:
1. Efficient synthetic methodologies for the preparation of αvβ3 target-specific photosensitizers.

(a) RGD conjugated photosensitizers

(b) Integrin-antagonist conjugated photosensitizers.
2. Multimodality agents (photosensitizer-cyanine dye conjugates) with and without RGD peptide.
3. Target-specific PET/fluorescence imaging agent.

### DETAILED DESCRIPTION OF THE INVENTION

As previously discussed, the invention is a compound that is a conjugate of an antagonist to an integrin expressed by a tumor cell and at least one of a fluorescent dye, and a tumor avid tetrapyrollic photosensitizer that may be complexed with an element X where X is a metal selected from the group consisting of Zn, In, Ga, Al, or Cu or a radioisotope labeled moiety wherein the radioisotope is selected from the group consisting of ¹¹C, ¹⁸F, ⁶⁴Cu, ¹²⁴I, ⁹⁹Tc, ¹¹¹In and GdIII and its method of use for diagnosing, imaging and/or treating hyperproliferative tissue such as tumors and other uncontrolled growth tissues such as found in macular degeneration.

In the case of the presence of a tetrapyrollic photosensitizer, it usually has the structural formula:

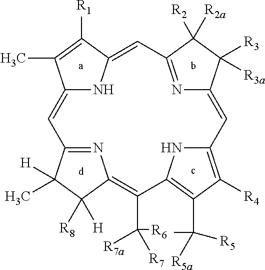

and its complexes with X where:
R₁ is —CH═CH₂, —CH₂CH₃, —CHO, —COOH, or

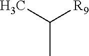

where R₉═—OR₁₀ where R₁₀ is lower alkyl of 1 through 8 carbon atoms, —(CH₂—O)ₙCH₃, —(CH₂)₂CO₂CH₃, —(CH₂)₂CONHphenyleneCH₂DTPA, —CH₂CH₂CONH(CONHphenyleneCH₂DTPA)₂, —CH₂R₁₁ or

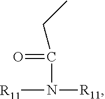

or a fluorescent dye moiety; R₂, R₂ₐ, R₃, R₃ₐ, R₄, R₅, R₅ₐ, R₇, and R₇ₐ are independently hydrogen, lower alkyl or substituted lower alkyl or two R₂, R₂ₐ, R₃, R₃ₐ, R₅, R₅ₐ, R₇, and R₇ₐ groups on adjacent carbon atoms may be taken together to form a covalent bond or two R₂, R₂ₐ, R₃, R₃ₐ, R₅, R₅ₐ, R₇, and R₇ₐ groups on the same carbon atom may form a double bond to a divalent pendant group; R₂ and R₃ may together form a 5 or 6 membered heterocyclic ring containing oxygen, nitrogen or sulfur; R₆ is —CH₂—, —NR₁₁— or a covalent bond; R₈ is —(CH₂)₂CO₂CH₃, —(CH₂)₂CONHphenyleneCH₂DTPA, —CH₂CH₂CONH(CONHphenyleneCH₂DTPA)₂, —CH₂R₁₁ or

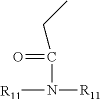

where
R₁, is —CH₂CONH-RGD-Phe-Lys, —CH₂NHCO-RGD-Phe-Lys, a fluorescent dye moiety, or —CH₂CONHCH₂CH₂SO₂NHCH(CO₂)CH₂NHCOPhenylOCH₂CH₂NHcycloCNH(CH₂)₃N; and polynuclide complexes thereof; provided that the compound contains at least one integrin antagonist selected from the group consisting of —CH₂CONH-RGD-Phe-Lys, —CH₂NHCO-RGD-Phe-Lys and
—CH₂CONHCH₂CH₂SO₂NHCH(CO₂)CH₂NHCOPhenylOCH₂CH₂NHcycloCNH(CH₂)₃N, where X is a metal selected from the group consisting of Zn, In, Ga, Al, or Cu or a radioisotope labeled moiety wherein the radioisotope is selected from the group consisting of ¹¹C, ¹⁸F, ⁶⁴Cu, ¹²⁴I, ⁹⁹Tc, ¹¹¹In and GdIII.

The complexes with X are readily made simply by heating the compound with a salt of X such as a chloride. The complex will form as a chelate of a -DTPA moiety, when present, or within the tetrapyrollic structure between the nitrogen atoms of the amine structure or both. Examples of such structures are:

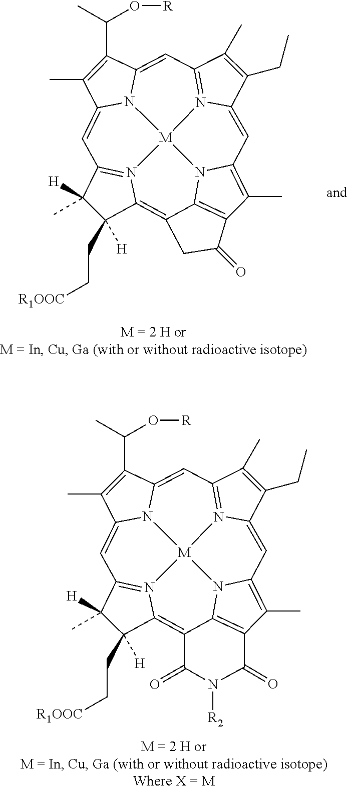

In the instance where a fluorescent dye is conjugated with the integrin antagonist (often a ligand), the fluorescent dye may be any non-toxic dye that causes the conjugate to preferentially emit (fluoresce) at a wave length of 800 to about 900 nm, e.g. indocyanine dyes. Such dyes usually have at least two resonant ring structures, often chromophores, connected together by an intermediate resonant structure of conjugated double bonds, aromatic carbon rings, resonant heterocylic rings, or combinations thereof.

Examples of such dyes include bis indole dyes wherein two indole or modified indole ring structures are connected together at their 3² and 2¹ carbon atoms respectively by an intermediate resonant structure as previously described. Such dyes are commonly known as tricarboclyanine dyes. Such dyes almost always have at least one, and usually at least two, hydrophilic substituents making the dye water soluble. Such water solubility facilitates entry of the structure into an organism and its cellular structures and reduces the likelihood of toxicity because of reduced storage in fatty tissues and fast elimination from the system. The intermediate resonant structure usually contains a plurality of double bonded carbon atoms that are usually conjugated double bonds and may also contain unsaturated carboxylic or heterocyclic rings. Such rings permit conjugation to a porphyrin or other structure without significantly interfering with the resonance of the intermediate structure. A preferred dye is indocyanine green.

When a radioisotope is combined with the integrin antagonist, it may be chemically combined by covalent or semi-ionic bonding or may be chelated into the compound. In such instances, the compound often includes known chelating structures such as DTPA.

### Preparation of 17²(17⁵-N-t-Bu-ethylene-diamido)Pyropheophorbide-a 2

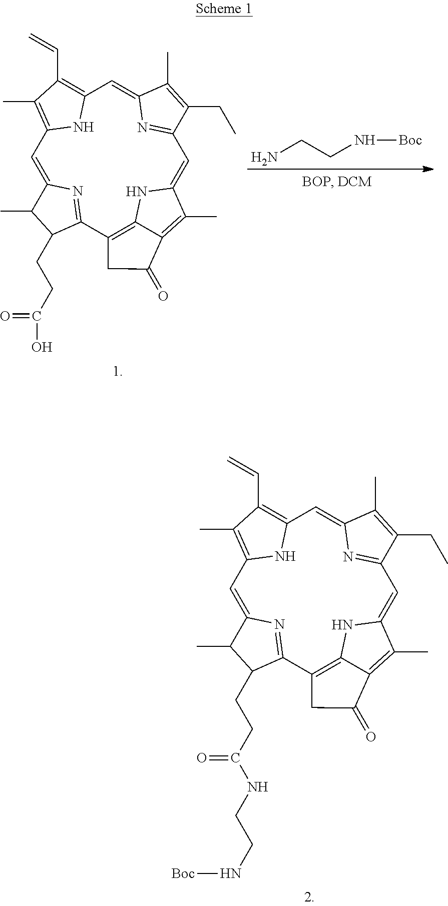

Pyropheophorbide-a carboxylic acid 1 (200 mg) was obtained from spirolina algae by following the literature procedure. It was dissolved in dry dichloromethane (DCM) (5 ml), to this solution under N₂ were added in sequence triethylamine (0.3 ml), Boc-protected diethylamine (66.6 ul) and BOP (146 mg), after evacuation (2-3 times), reaction mixture was stirred at room temperature for overnight under N₂. Reaction mixture was concentrated and chromatographed on silica (eluent: 4% Methanol in dichloromethane) and the desired compound 2 was isolated as the major product. Yield 90%. NMR (AMX400): (CDCl₃, δ ppm): 9.35, 9.15 and 8.50 (each s, 1H, meso H); 7.80 (m, 1H CH═CH₂); 6.25, 6.1 (each d, 1H, CH—CH₂); 5.22(dd, 2H, —CH₂ exocyclic ring); 4.41(q, 1H,18H); 4.28 (d,1H, 17H); 3.75 (q,2H,CH₂—CH₃); 3.62, 3.4, 3.25 (each s, 3H, ring —CH₃), 2.8-2.0 (several m, CH₂—CH₂—CO—NH—CH₂—CH₂—NH), 1.2 (s, 9H, Boc).

### Preparation of Pyropheophorbide-Cyclo(Lys-Arg-Gly-Asp-L-Phe) Conjugate

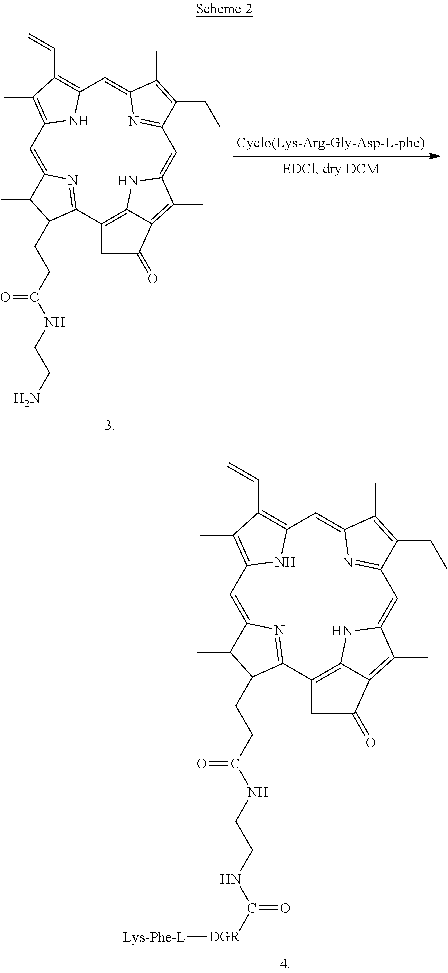

Pyropheophorbide 2 was treated with 90% trifluroacetic acid (TFA) to remove Boc group, TFA was removed on rotaevaporator and 3 was dried under high vacuum for further reaction. 3 (15 mg) was dissolved in dry DCM, to this solution were added under N₂ Cyclo(Lys-Arg-Gly-Asp-L-Phe) (20 mg) and EDCI (12 mg), reaction mixture was stirred at room temperature for overnight under N₂. Reaction mixture was concentrated and chromatographed on preparative silica plate (eluent: 10% Methanol in dichloromethane). The isolated compound was further treated with 90% TFA/DCM for 3-4 hrs. to get the desired pyropheophorbide . . . 4. TFA was rotaevaporated and the compound was further purified on HPLC using C-18 column (eluent: gradient 90% MeOH in water to 100% MeOH in water, flow rate 0.5 ml/min). Yield 10 mg. Mass: m/z=1161 (M+H)⁺.

### Preparation of meso-Purpurinimide 6

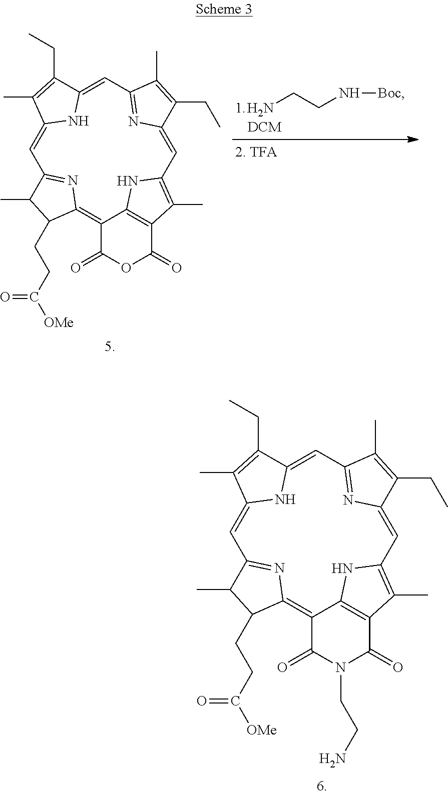 [SMILES: CCC1=C(C)c2cc3[nH]c(cc4nc(c5c6[nH]c(cc1n2)c(C)c6C(=O)OC5=O)C(CCC(=O)OC)C4C)c(C)c3CC ; CC(C)(C)OC(=O)NCCN ; CCC1=C(C)c2cc3[nH]c(cc4nc(c5c6[nH]c(cc1n2)c(C)c6C(=O)N(CCN)C5=O)C(CCC(=O)OC)C4C)c(C)c3CC]

Meso-purpurinimide (60 mg) and Boc-protected diethylamine (2.24 g) were dissolved in minimum amount of DCM and the reaction mixture was stirred for 48 hrs at room temperature under N₂. UV-VIS showed the complete shift of absorbance from 685 nm to 651 nm. To this reaction mixture, freshly prepared diazomethane (200-400 mg) was added and the reaction was monitored by TLC (5% MeOH in DCM). After 10-min UV-VIS showed the complete disappearance of peak at 651 nm and the product peak at 695 nm. Reaction mixture was immediately washed with 2% acetic acid in water and then with water (×3), compound was dried on Na₂SO₄, concentrated and chromatographed on silica (eluent: 2-3% Methanol in dichloromethane), the isolated compound was further treated with 90% TFA/DCM for 3-4 hrs, TFA was rotaevaporated to get the desired compound 6 as the major product. Yield 90%. NMR (AMX400): 9.54 (s, 1H, 10H); 9.16 (s, 1H, 5H); 8.4 (s, 1H, 20H); 5.34 (m, 1H,17H), 4.67 (m, 2H, N—CH₂), 4.34(q, 1H, 18H), 3.78, 3.58, 3.23, 3.15 (each, 3H, 12CH₃, 17² CH₃, 2CH₃, 7CH₃ resp.) 3.74 (q,2H, 8′CH₂), 3.605 (CH₂—CH₃), 2.71 (m, 1H, 1×17²H), 2.402 (m, 2H, 2×17′H), 2.0 (m,1H, 17²H), 1.76 (d, 3H, 18CH₃), 1.7-1.64 (8H, 8² CH₂—CH₃, 3 CH₂—CH₃, N—CH₂—CH₂—NH₂), 0.11-0.1 (2H, each s, —NH).

### Preparation of meso-Purpurinimide-Cyclo ((Lys-Arg-Gly-Asp-L-Phe) Conjugate 8

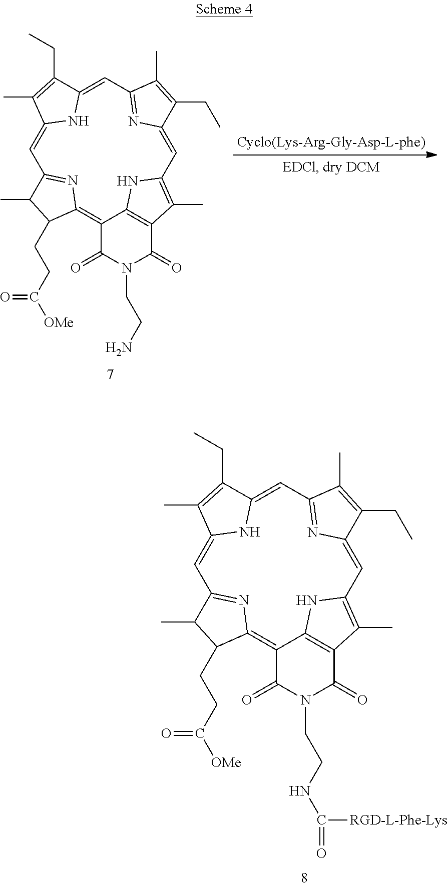

Meso-Purpurinimide 6 (17 mg) was dissolved in dry DCM, to this solution were added under N₂ Cyclo(Lys-Arg-Gly-Asp-L-Phe) (20 mg) and EDCI (12 mg), reaction mixture was stirred at room temperature for overnight under N₂. Reaction mixture was concentrated and chromatographed on preparative silica plate (eluent: 10% Methanol in dichloromethane). The isolated compound was further treated with 90% TFA/DCM for 3-4 hrs. to get the desired meso-Purpurinimide-Cyclo((Lys-Arg-Gly-Asp-L-Phe) conjugate 8. TFA was rotaevaporated and the compound was dried under high vacuum. Yield 19 mg. Mass: m/z=1207 (M+H)⁺

### Preparation of Pyropheophorbide-Cyclo(Lys-Arg-Gly-Asp-D-Phe) Conjugate 8

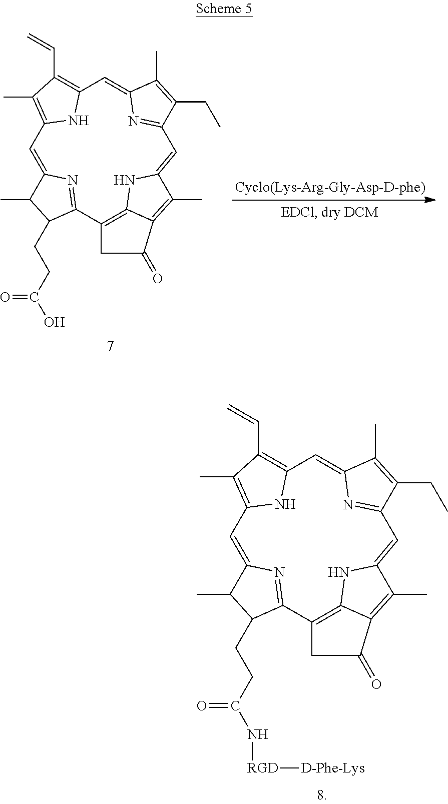

Pyropheophorbide-a carboxylic acid 7 (200 mg) was obtained from spirolina algae by following the literature procedure. 7(14 mg) was dissolved in dry DCM, to this solution were added under N₂ Cyclo(Lys-Arg-Gly-Asp-D-Phe) (20 mg), EDCI (12 mg) and DMAP (12 mg), reaction mixture was stirred at room temperature for overnight under N₂. Reaction mixture was concentrated and chromatographed on preparative silica plate (eluent: 10% Methanol in dichloromethane). The isolated compound was further treated with 90% TFA/DCM for 3-4 hrs. and the solid product was washed with MeOH to get the desired pyropheophorbide-Cyclo(Lys-Arg-Gly-Asp-D-Phe) conjugate 8, TFA was rotaevaporated and the compound was dried under vacuum. Yield 10 mg. Mass: m/z=1119.6 (M+H)⁺

### Preparation of meso-Purpurinimide-glycine ester 10

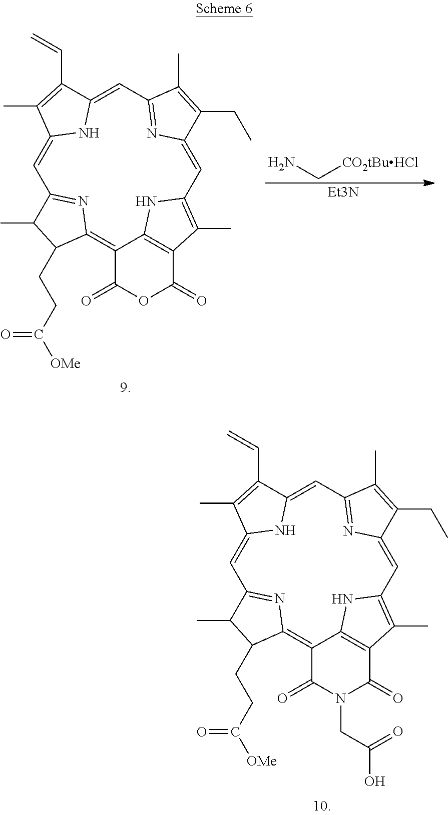

58 mg of purpurin-18 was dissolved in minimum amount of toluene, to this solution HCl salt of glycine-t-Bu ester and 10-15 drops of triethylamine were added, reaction was refluxed under N₂, after 3 hrs UV-VIS showed the complete disappearance of peak at 696 nm of starting material and new peak at 705 nm, Reaction mixture was concentrated and chromatographed on silica (eluent: 2% Methanol in dichloromethane). and the desired meso-Purpurinimide-glycine ester 10 was isolated as the major product. Yield 90%. NMR (AMX400): 9.64 (s, 1H, 10H), 9.39 (s, 1H, 15H), 8.58 (s,1H, 20H), 7.84 (d, 1H, 3CH—CH₂), 6.16 (d,1H, 3CH═CH₂), 5.4(m,1H,17H), 4.46 (m, 2H, N—CH₂—CH₂—CO₂H), 4.31 (q, 1H, 18H), 3.84 (s, 3H, 7CH₃); 2.68 and 2.39 (each m, 1H+2H, 2×17¹H); 1.99 (m, 1H, 1×17²H); 1.74 (d, 3H, 18CH₃), 1.64 (t, 3H, 8² CH₃); 0.07 and −0.16 (each br, 1H, 2NH).

### Preparation of meso-Purpurinimide-glycine-Cyclo(Lys-Arg-Gly-Asp-D-Phe) Conjugate 12

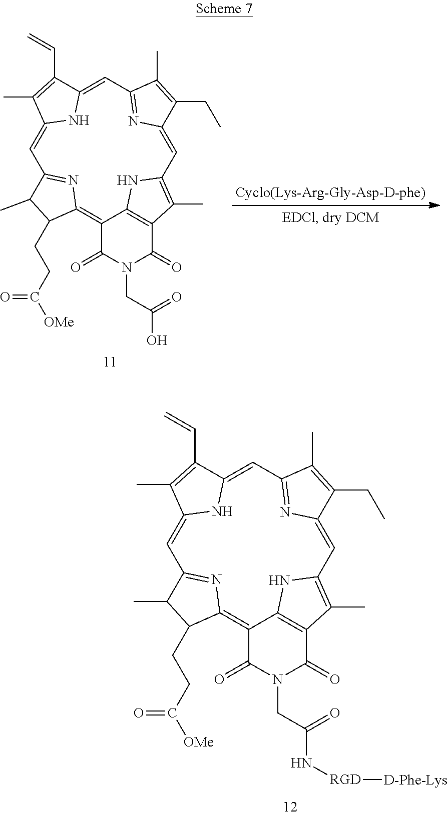

MMeso-Purpurinimide-glycine ester 10 (17 mg) was dissolved in dry DCM, to this solution were added under N₂ Cyclo(Lys-Arg-Gly-Asp-D-Phe) (20 mg), EDCI (12 mg) and DMAP (12 mg), reaction mixture was stirred at room temperature for overnight under N₂. Reaction mixture was concentrated and the solid powder was washed with MeOH. The isolated compound was further treated with 90% TFA/DCM for 3-4 hrs. to get the desired meso-Purpurinimide-glycine-Cyclo(Lys-Arg-Gly-Asp-D-Phe) conjugate 12, TFA was rotaevaporated, washed with MeOH and dried under vacuum. Yield 20 mg. Mass: m/z=1220 (M+H)⁺.

### Preparation of Mono-I-Cypate

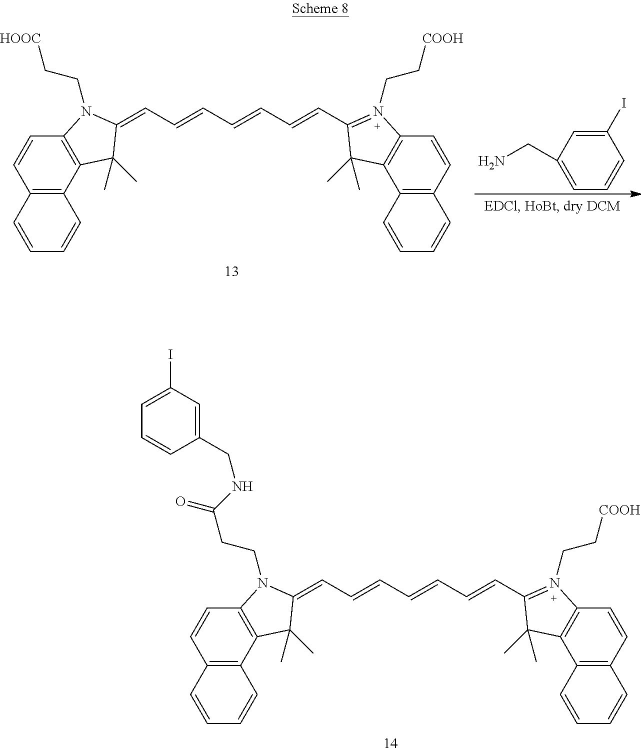

Cypate 13 (260 mg, 0.4 mM) was dissolved in dry DMF (10-15 ml), to this solution were added under N₂ m-I-benzylamine (92 mg, 0.4 mM), EDCI (92 mg, 0.48 mM) and HoBt(64.75 mg, 0.48 mM), reaction mixture was stirred at room temperature for overnight under N₂. After overnight reaction, DMF was removed under high vacuum, reaction mixture was washed with brine (×3) and water (×3), dried over Na₂SO₄ and concentrated. Purification was done on Si column using MeOH/DCM as an eluant. Yield 57 mg (17%). Mass: m/z=839 (M+H)⁺. NMR (AMX400): 7.25-8.03 (m, 16H, aromatic), 6.28-6.80 (m, 4H, —CH), 2.47-3.0 (m, 10H, CH₂), 1.88 (s, 12H, CH₃).

### Preparation of Mono-I-Cypate-Cyclo(Lys-Arg-Gly-Asp-D-Phe) Conjugate 16

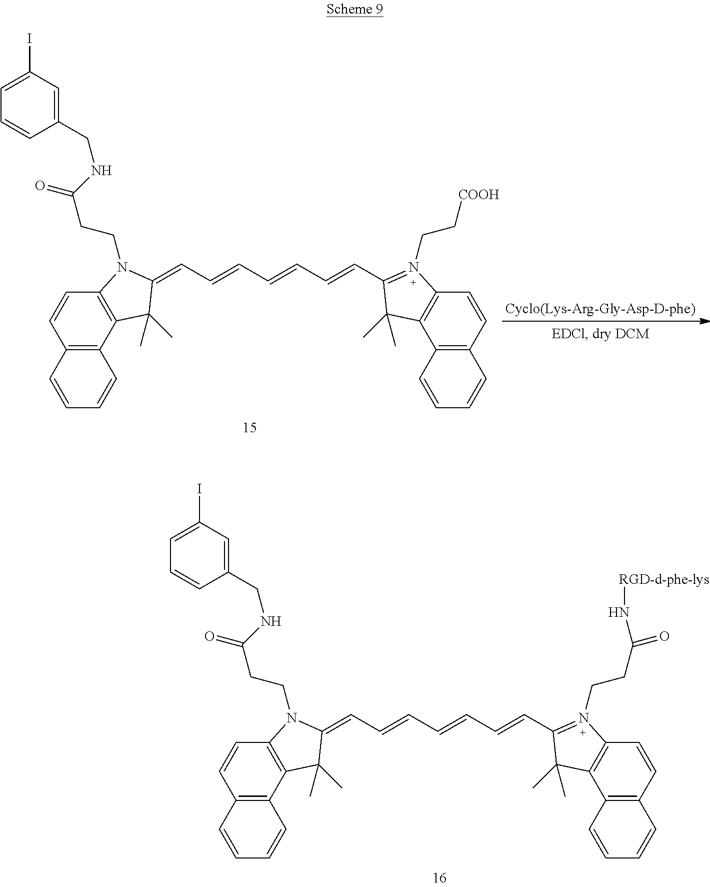

Mono-I-Cypate(30 mg) was dissolved in dry DCM, to this solution were added under N₂ Cyclo(Lys-Arg-Gly-Asp-D-Phe) (20 mg), EDCI (12 mg) and DMAP (12 mg), reaction mixture was stirred at room temperature for overnight under N₂. After overnight stirring, reaction mixture was concentrated and chromatographed on preparative silica plate (eluent: 13% Methanol in Dichloromethane). The isolated compound was further treated with 90% TFA/DCM for 3-4 hrs. and the oily product was further analyzed and purified on an HPLC (Waters, Delta 600 with 996 photodiode array detector) Ana. Column: Waters Symm-C-81, 4.6×150 mm, 5μ: Semiprep Column: Waters Symm-C-18, 7.8×150 mm, 7μ: using Acetinitrile/Water as an eluant (gradient: 30% to 100% ACN) to get the desired mono-I-Cypate-Cyclo(Lys-Arg-Gly-Asp-D-Phe) conjugate 16, Yield 24 mg. Mass: m/z=1424 (M+H)⁺.

### Pyro-IA (methyl ester)(19)

To a solution of Methyl 3-[4-{2-(3,4,5,6-tetrahydropyrimidin-2-ylamino)ethyloxy}-benzoyl]amino-2-(S)-aminoethylsulfonylaminopropionate (17) (47 mg, 0.1 mmol) and pyrocarboxylic acid (18) (60 mg, 0.11 mmol) in anhydrous DMF (5.0 mL) under nitrogen atmosphere, PyBOP (65 mg, 0.12 mmol) and anhydrous triethylamine (0.3 mL) was added and resultant reaction mixture was stirred for overnight at room temperature. Reaction mixture was then rotary evaporated down to dryness and desired product (19) was obtained after purifying crude reaction mixture first over prep silica TLC plate (eluant: 10% MeOH in CH2C12) followed by short silica column (eluant: 8% MeOH in CH2C12). Yield=50 mg (50%)

¹H-NMR(10% CD₃OD in CDCl₃; 400 MHz): δ 9.39, 9.28 and 8.56(all s, 1H, meso-H); 7.95(dd, J=11.4, 18.2, 1H, 3-vinyl); 7.73(d, J=8.8, 2H, ArH); 6.84(d, J=8.8, 2H, ArH); 6.28(d, J=17.6, 1H, 3-vinyl); 6.18(d, J=11.6, 1H, 3-vinyl); 5.26(d, J=20, 1H, 13²-CH₂); 5.06(d, J=20, 1H, 13²-CH₂); 4.51(m, 1H, 18-H); 4.30-4.20(m, 2H, CH & 17-H); 4.00(t, J=5.0, 2H, OCH₂); 3.85(m, 1H, CONHCH₂); 3.67 (s, 3H, ring CH₃); 3.62(m, 2H, 8-CH₂CH₃); 3.60(m, 1H, CONHCH₂); 3.58(s, 3H, OCH₃); 3.42(t, J=5.0, 2H, SO₂CH₂); 3.38(s, 3H, ring CH₃); 3.37-3.31(m, 6H, 3×NHCH₂); 3.19(s, 3H, ring CH₃); 3.14(m, 2H, 3×NCH₂); 2.66, 2.45, 2.28, 2.20 (all m, 4H, 17′ and 17²-H); 1.93(t, J=5.6, 2H, CH₂); 1.80(d, J=7.2, 3H, 18-CH₃); 1.68(t, J=7.8, 3H, 8-CH₂CH₃). Mass for C₅₂H₆₂N₁₀O₈S : 986.45 (Calculated); 986.6 (Found, M⁺).

### Pyro-Integrin Antagonist-IA (20)

To a solution of Pyro-IA (methyl ester) (19)(40 mg) in dry THF (10 mL) under argon atmosphere, a solution of LiOH (80 mg, in 5+4 mL: H2O+MeOH respectively) was added and reaction mixture was stirred for 45 min. Reaction was then carefully neutralized with cation exchange resin. Resin was filtered out and reaction mixture was rotary evaporated down to dryness. No further attempt was made to purify the product.

Yield=35 mg (90%). ¹H-NMR(25% CD₃OD in CDCl₃; 400 MHz): δ 9.39, 9.28 and 8.56(all s, 1H, meso-H); 7.95(dd, J=11.4, 18.2, 1H, 3-vinyl); 7.73(d, J=8.8, 2H, ArH); 6.84(d, J=8.8, 2H, ArH); 6.28(d, J=17.6, 1H, 3-vinyl); 6.18(d, J=11.6, 1H, 3-vinyl); 5.26(d, J=20, 1H, 13²-CH₂); 5.06(d, J=20, 1H, 13²-CH₂); 4.51(m, 1H, 18-H); 4.30-4.20(m, 2H, CH & 17-H); 4.00(t, J=5.0, 2H, OCH₂); 3.85(m, 1H, CONHCH₂); 3.67 (s, 3H, ring CH₃); 3.62(m, 2H, 8-CH₂CH₃); 3.60(m, 1H, CONHCH₂); 3.42(t, J=5.0, 2H, SO₂CH₂); 3.38(s, 3H, ring CH₃); 3.37-3.31(m, 6H, 3×NHCH₂); 3.19(s, 3H, ring CH₃); 3.14(m, 2H, 3×NCH₂); 2.66, 2.45, 2.28, 2.20 (all m, 4H, 17¹ and 17²-H); 1.93(t, J=5.6, 2H, CH₂); 1.80(d, J=7.2, 3H, 18-CH₃); 1.68(t, J=7.8, 3H, 8-CH₂CH₃). Mass for C₅₂H₆₂N₁₀O₈S: 972.4 (Calculated); 972.6 (Found, M⁺).

### Purpurinimide-Gly-IA (methyl ester)(22)

To a solution of Methyl 3-[4-{2-(3,4,5,6-tetrahydropyrimidin-2-ylamino)ethyloxy}-benzoyl]amino-2-(S)-aminoethylsulfonylaminopropionate (17) (20 mg, 0.04 mmol) and glycine purpurinimide (21) (20 mg, 0.03 mmol) in anhydrous DMF (3.0 mL) under nitrogen atmosphere, PyBOP (20 mg, 0.04 mmol) and anhydrous triethylamine (0.1 mL) was added and resultant reaction mixture was stirred for overnight at room temperature. Reaction mixture was then rotary evaporated down to dryness and desired product (22) was obtained after purifying crude reaction mixture first over prep silica TLC plate (eluant: 10% MeOH in CH2C12) followed by short silica column (eluant: 8% MeOH in CH2C12). Yield=15 mg (45%)

¹H-NMR(10% CD₃OD in CDCl₃; 400 MHz): δ 9.07, 8.94 and 8.58(all s, 1H, meso-H); 7.82(dd, J=11.4, 18.2, 1H, 3-vinyl); 7.70(d, J=8.8, 2H, ArH); 6.75(d, J=8.8, 2H, ArH); 6.26(d, J=17.6, 1H, 3-vinyl); 6.16(d, J=11.6, 1H, 3-vinyl); 5.25(d, J=7.2, 1H, 17-H); 5.10(dd, J=8.6, 16.0, 2H, NCH₂); 4.42(dd, J=4.4, 7.6, 1H, CH); 4.35(q, J=6.8, 1H, 18-H); 3.89(m, 2H, OCH₂); 3.85(m, 1H, CONHCH₂); 3.80 (m, 2H, NHCH₂); 3.72, 3.52, 3.36, 3.33 and 2.85(all s, all 3H, for 3× ring CH₃ & 2×OCH₃); 3.67(m, 1H, CONHCH₂); 3.35(m, 4H, 2×NHCH₂); 3.26 (m, 4H, 8-CH₂CH₃ and SO₂CH₂); 3.15(m, 2H, NCH₂); 3.62(m, 2H, 8-CH₂CH₃); 2.68, 2.38, 1.98 (all m, 4H, 17¹ and 17²-H); 1.83(t, J=5.6, 2H, CH₂); 1.80(d, J=7.2, 3H, 18-CH₃); 1.41(t, J=7.8, 3H, 8-CH₂CH₃). Mass for C₅₅H₆₅N₁₁O₁₁S: 1087.46 (Calculated); 1087.8 (Found, M⁺).

### Purpurinimide-Gly-IA (23)

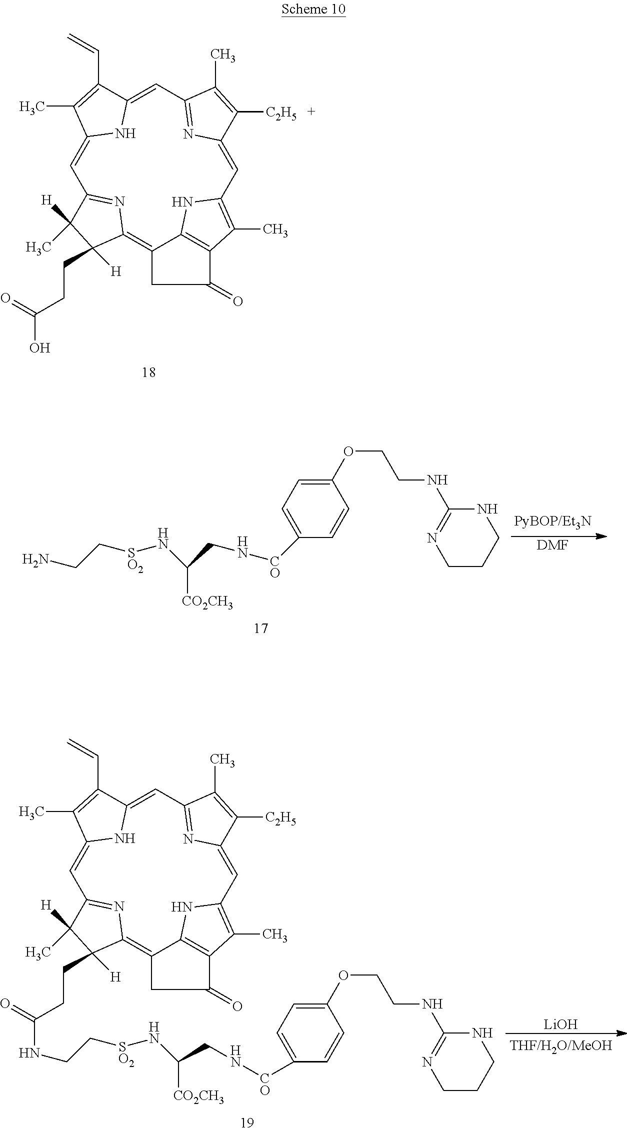

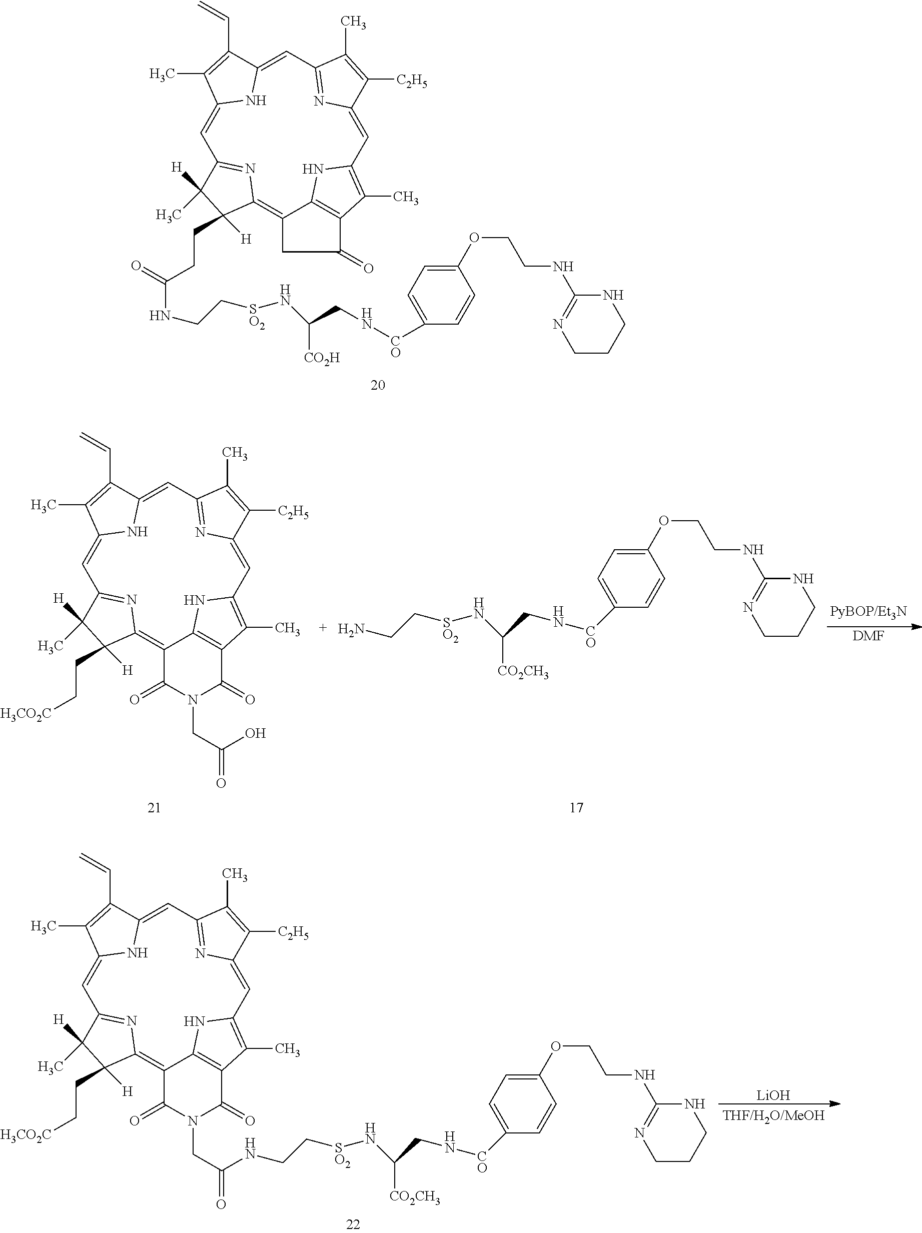

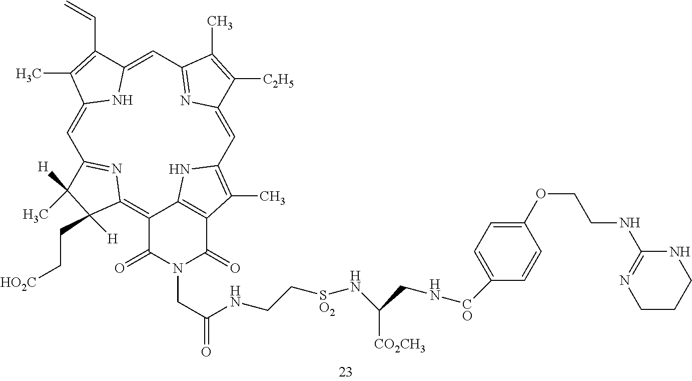 [SMILES: C=Cc1c(C)c2cc3nc(c4c5[nH]c(cc6nc(cc1[nH]2)C(C)=C6CC)c(C)c5C(=O)N(CC(=O)NCCS(=O)(=O)N[C@@H](CNC(=O)c1ccc(OCCNC2=NCCCN2)cc1)C(=O)OC)C4=O)[C@@H](CCC(=O)O)[C@@H]3C]

To a solution of Purpurinimide-Gly-IA (methyl ester)(22) (15 mg) in dry THF (7 mL) under argon atmosphere, a solution of LiOH (30 mg, in 4+3 mL: H2O+MeOH respectively) was added and reaction mixture was stirred for 45 min. Reaction was then carefully neutralized with cation exchange resin. Resin was filtered out and reaction mixture was rotary evaporated down to dryness. No further attempt was made to purify the product. Yield=12 mg (85%)

¹H-NMR(25% CD₃OD in CDCl₃; 400 MHz): δ 9.07, 8.94 and 8.58(all s, 1H, meso-H); 7.82(dd, J=11.4, 18.2, 1H, 3-vinyl); 7.70(d, J=8.8, 2H, ArH); 6.75(d, J=8.8, 2H, ArH); 6.26(d, J=17.6, 1H, 3-vinyl); 6.16(d, J=11.6, 1H, 3-vinyl); 5.25(d, J=7.2, 1H, 17-H); 5.10(dd, J=8.6, 16.0, 2H, NCH₂); 4.42(dd, J=4.4, 7.6, 1H, CH); 4.35(q, J=6.8, 1H, 18-H); 3.89(m, 2H, OCH₂); 3.85(m, 1H, CONHCH₂); 3.80 (m, 2H, NHCH₂); 3.36, 3.33 and 2.85(all s, all 3H, for 3× ring CH₃); 3.67(m, 1H, CONHCH₂); 3.35(m, 4H, 2×NHCH₂); 3.26 (m, 4H, 8-CH₂CH₃ and SO₂CH₂); 3.15(m, 2H, NCH₂); 3.62(m, 2H, 8-CH₂CH₃); 2.68, 2.38, 1.98 (all m, 4H, 17¹ and 17²-H); 1.83(t, J=5.6, 2H, CH₂); 1.80(d, J=7.2, 3H, 18-CH₃); 1.41(t, J=7.8, 3H, 8-CH₂CH₃). Mass for C₅₅H₆₅N₁₁O₁₁S: 1059.43 (Calculated); 1059.8 (Found, M⁺).

What is claimed is:

1-12. (canceled)

13. A compound consisting essentially of a conjugate of:
a) an RGD peptide antagonist to an integrin receptor expressed by a tumor cell,
b) a photosensitizer comprising a bacteriochlorin, a purpurinimide or a pyropheophorbide ring, and
c) X where X is a radioisotope labeled moiety wherein the radioisotope consists of ¹⁸F.

14. A compound of claim 13 wherein the photosensizer is a pyropheophorbide-a.

15. A compound of claim 13 comprising the conjugate of the X labeled moiety with a photosensitizer-RGD peptide structure having the formula:

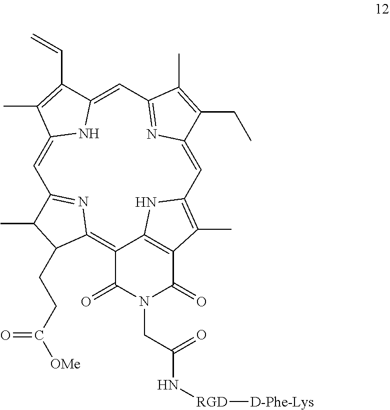

16. A compound of claim 13 comprising the conjugate of the X labeled moiety with a photosensitizer-RGD peptide structure having the formula:

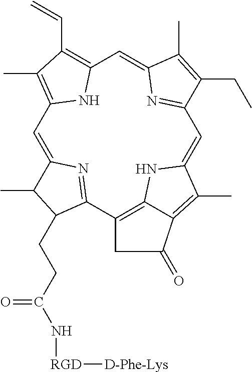

17. A compound of claim 13 comprising the conjugate of the X labeled moiety with a photosensitizer-RGD peptide structure having the formula:

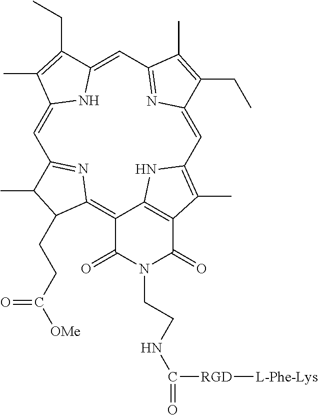

18. A compound of claim 13 comprising the conjugate of the X labeled moiety with a photosensitizer-RGD peptide structure having the formula: [chemical structure]
